(19) United States
(12) Patent Application Publication
(10) Pub. No.: US 2026/0179720 A1
(43) Pub. Date: Jun. 25, 2026

(54) SYSTEMS AND METHODS FOR DETECTING FUSION GENES FROM SEQUENCING DATA

(71) Applicants: UNIVERSITY OF WASHINGTON, Seattle, WA (US); FRED HUTCHINSON CANCER CENTER, Seattle, WA (US)

(72) Inventors: Ka Yee Yeung-Rhee, Seattle, WA (US); Ling-Hong Hung, Seattle, WA (US); Shishir Reddy, Seattle, WA (US); Olga Sala-Torra, Seattle, WA (US); Cecilia Yeung, Seattle, WA (US)

(73) Assignees: UNIVERSITY OF WASHINGTON, Seattle, WA (US); FRED HUTCHINSON CANCER CENTER, Seattle, WA (US)

(21) Appl. No.: 19/121,347

(22) PCT Filed: Oct. 13, 2023
(86) PCT No.: PCT/US2023/076915

§ 371 Date: Apr. 15, 2025

Related U.S. Application Data
(60) Provisional application No. 63/416,888, filed on Oct. 17, 2022.

(51) Int. Cl. G16B 20/00; C12Q 1/6869; G16B 30/00

(52) U.S. Cl. CPC G16B 20/00; C12Q 1/6869; G16B 30/00

(57) ABSTRACT

In some embodiments, a computer-implemented method of detecting a presence of a predetermined fusion gene in a biological sample is provided. A computing system generates an alignment of a read sequence to a reference genome. The alignment includes a first alignment result and a second alignment result. The computing system determines a breakpoint location indicated by the first alignment result and the second alignment result, distances between coordinates of the breakpoint location and coordinates of one or more expected breakpoint locations associated with the predetermined fusion gene, a gap size value and an overlap size value. In response to determining that the gap size value is less than a gap size value threshold, the overlap size value is less than an overlap size value threshold, and the distances are less than a breakpoint distance threshold, the computing system generates an indication of the presence of the predetermined fusion gene.

### CROSS-REFERENCE(S) TO RELATED APPLICATION(S)

This application claims the benefit of Provisional Application No. 63/416,888, filed Oct. 17, 2022, the entire disclosure of which is hereby incorporated by reference herein for all purposes.

### STATEMENT OF GOVERNMENT LICENSE RIGHTS

This invention was made with government support under CA280520 awarded by the National Institutes of Health. The government has certain rights in the invention.

### BACKGROUND

Fusion genes are hybrid genes that are formed when two genes that were previously independent are rearranged, and typically occur due to a translocation, interstitial deletion, or chromosomal inversion. Many diagnostic tests, including but not limited to classification of myeloid malignancies, are largely based on the detection of molecular and genetic aberrations. For example, current gene rearrangements are present in 30-40% of acute myeloidleukemia (AML), and well-described driver fusions sometimes suffice to diagnose leukemias (for example, PML::RARA, or RUNX1::RUNX1T1 among others). These fusion genes confer specific clinical and biological characteristics as drivers of leukemogenesis that can assist in prognosis stratification and inform treatment decisions.

While the rise of inexpensive, fast, widely available sequencing technologies such as nanopore sequencing has greatly increased the ability to generate sequencing information for samples obtained from subjects, there are nevertheless technical problems with using this sequencing information to detect the presence of fusion genes. For example, though they are fast, these sequencing technologies have a tendency to produce some errors in sequencing due to a variety of factors, including but not limited to uncertainty in the basecalling process. Previous attempts at detecting fusion genes (such as LongGF) that only detected fusion genes if the breakpoints were exactly matched were highly sensitive to these errors, and tended to miss fusion genes present in this type of sequencing information.

What is desired are computing techniques that overcome these technical issues to be able to successfully detect fusion genes in sequencing information that may include occasional misalignments or other errors.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In some embodiments, a computer-implemented method of detecting a presence of a predetermined fusion gene in a biological sample is provided. A computing system receives a read sequence, and generates an alignment of the read sequence to a reference genome. The alignment includes a first alignment result and a second alignment result, wherein the first alignment result indicates an alignment of a first portion of the read sequence to a first location in the reference genome, and the second alignment result indicates an alignment of a second portion of the read sequence to a second location in the reference genome. The computing system determines a breakpoint location indicated by the first alignment result and the second alignment result, distances between coordinates of the breakpoint location and coordinates of one or more expected breakpoint locations associated with the predetermined fusion gene, a gap size value and an overlap size value based on the first portion of the read sequence and the second portion of the read sequence. In response to determining that the gap size value is less than a gap size value threshold, the overlap size value is less than an overlap size value threshold, and the distances are less than a breakpoint distance threshold, the computing system generates an indication of the presence of the predetermined fusion gene.

In some embodiments, a computer-implemented method of detecting fusion genes in a biological sample is provided. A computing system receives a plurality of read sequences. For each read sequence of the plurality of read sequences, the computing system generates an alignment of the read sequence to a reference genome, wherein the alignment includes a first alignment result and a second alignment result, wherein the first alignment result indicates an alignment of a first portion of the read sequence to a first location in the reference genome, and wherein the second alignment result indicates an alignment of a second portion of the read sequence to a second location in the reference genome; determines a breakpoint location indicated by the first alignment result and the second alignment result; determines a gap size value and an overlap size value based on the first portion of the read sequence and the second portion of the read sequence; and in response to determining that the gap size value is less than a gap size value threshold and the overlap size value is less than an overlap size value threshold, adds the breakpoint location to a set of candidate breakpoint locations. The computing system determines a similar breakpoint set that includes breakpoint locations of the set of candidate breakpoint locations for which the breakpoint locations are within a threshold distance of each other, and in response to determining that a size of the similar breakpoint set is larger than a threshold, generates an indication of a detected fusion gene associated with the similar breakpoint set.

In some embodiments, a non-transitory computer-readable medium having computer-executable instructions stored thereon is provided. The instructions, in response to execution by one or more processors of a computing system, cause the computing system to perform actions of a method as described above.

In some embodiments, a computing system configured to perform actions of a method as described above is provided.

### BRIEF DESCRIPTION OF THE DRAWINGS

The foregoing aspects and many of the attendant advantages of this disclosure will become more readily appreciated as the same become better understood by reference to the following detailed description, when taken in conjunction with the accompanying drawings, wherein:

FIG. 1 is a schematic illustration of a system for nanopore-based analysis according to various aspects of the present disclosure.

FIG. 2 is a schematic illustration of a non-limiting example embodiment of a flow cell according to various aspects of the present disclosure.

FIG. 3 is a block diagram that illustrates aspects of a non-limiting example embodiment of a fusion finder computing system according to various aspects of the present disclosure.

FIG. 4 is a schematic illustration of a fictional fusion gene according to various aspects of the present disclosure.

FIG. 5A and FIG. 5B illustrate a first read sequence and a second read sequence that indicate the presence of the fusion gene illustrated in FIG. 4 without any errors.

FIG. 6 illustrates a third read sequence that should be considered as indicating the presence of the fusion gene illustrated in FIG. 4, but that includes a minor sequencing error that leads to a change in the detected breakpoint.

FIG. 7 illustrates a fourth read sequence that should be considered as indicating the presence of the fusion gene illustrated in FIG. 4, but that includes a minor sequencing error that leads to a gap in coverage of the fourth read sequence.

FIG. 8 illustrates another potential issue that may arise in attempting to detect fusion genes using read sequences.

FIG. 9A and FIG. 9B are a flowchart that illustrates a non-limiting example embodiment of a method of detecting one or more predetermined fusion genes indicated by sequencing data, according to various aspects of the present disclosure.

### DETAILED DESCRIPTION

Low-and middle-income countries (LMICs) suffer the heaviest burden of cancer deaths. Scarce pathology services that result in incomplete, inappropriate, or delayed diagnoses are one of the causes of higher morbidity and mortality rates. A slow or incorrect diagnosis can result in disease progression with worsening prognosis, or incorrect treatment decisions. A systematic review showed that earlier testing after the start of symptoms is associated with lower-stage disease and improved survival benefits for breast cancers, colorectal cancers, head and neck cancers, prostate cancers, and melanoma. In 2017, only ¼ of low-income countries reported having readily available access to pathology services. In response, the WHO provided guidance on early cancer diagnosis that emphasized early diagnosis to include access to disease evaluation to guide subsequent treatment.

Detection of diagnostic fusions (fusion genes associated with particular conditions) may be especially relevant in developing countries to guide the use of resources as they have several novel target therapies available since they are included in the WHO list of essential medicines. Examples include, but are not limited to, tyrosine kinase inhibitors (TKI) in chronic myelogenous leukemia (CML) that block BCR::ABL1 kinase activity and differentiation therapy with all-trans retinoic acid (ATRA) in acute promyelocytic leukemia (APL) targeting the (PML::RARA) fusion.

Typically, fusion genes are detected by fluorescence in situ hybridization (FISH), polymerase chain reaction (PCR) assays, or next-generation sequencing (NGS). NGS is an attractive technology because it should be able to detect a variety of genetic mutations found in leukemia, including fusions, insertions, deletions, and point mutations in oncogenic genes. However, the application of conventional NGS in this context suffers from technical hurdles (short sequencing reads, introduction of false positive errors due to library preparation) and hardware issues (expensive machines, complicated bioinformatics). These issues make NGS hard to employ readily in LMIC settings.

The advancement of long-read sequencing technologies has enabled the sequencing of continuous single DNA or RNA molecules up to tens to hundreds of kilobases (kb) long. Ongoing improvements in nanopore sequencing accuracy have reduced error rates to less than 5% but remain higher than those for Illumina and Ion Torrent platforms, which are used frequently in clinical laboratories. Long-read sequencing has made an impact on the understanding of the pathobiology of various diseases, and its impact should increase as the sequencing quality improves and becomes more accurate.

Addition of CRISPR-Cas9 for targeted enrichment concentrates the regions of interest prior to sequencing by nanopore without requiring amplification steps, thus optimizing sequencing time and efficiency. The portability and affordability of the Nanopore sequencer MinION and Flongle hold great promise to impact the clinical field, especially in the LMIC setting. However, a major limitation has been the lack of analytical software featuring standardized parameters to aid in translation into clinical diagnostics. Implementing such a portable platform with reduced cost and cloud-based on-demand analysis workflow, particularly in developing countries, would enable testing centers to provide sequencing without investing in a computing or bioinformatics infrastructure, thus bridging the gap between diagnosis and tailored treatment administration.

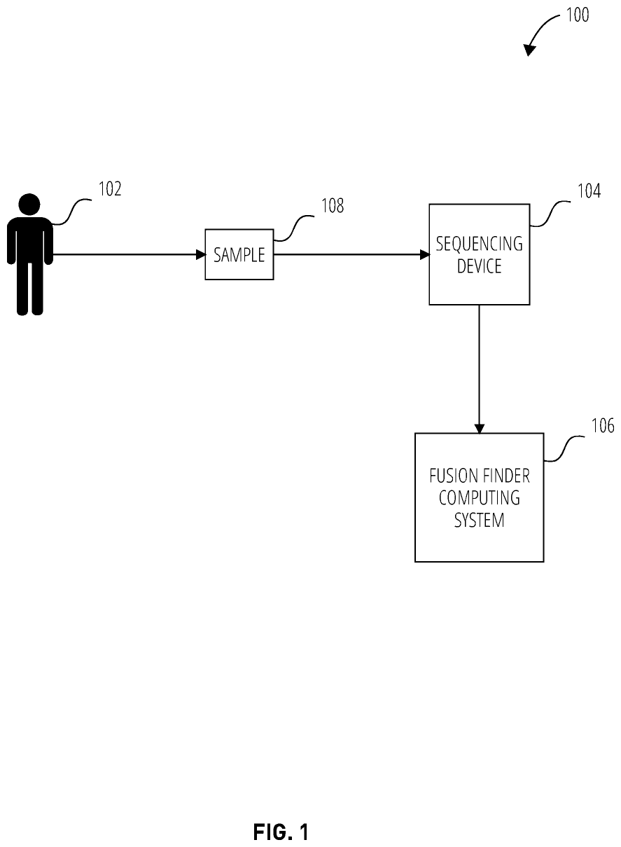

FIG. 1 is a schematic illustration of a system for nanopore-based analysis according to various aspects of the present disclosure. As shown, in the system 100, a sample 108 is obtained from a subject 102 using known techniques. The sample 108 may be a tissue biopsy, a swab, a blood sample, or any other suitable type of sample 108. The sample 108 is prepared (e.g., combined with one or more buffers, enzymes, etc.), and the prepared sample 108 is provided to a sequencing device 104, such as a flow cell. One non-limiting example of a sequencing device 104 is a MinION sequencing device provided by Oxford Nanopore Technologies plc. Some non-limiting examples of devices used with the sequencing device 104, such as a flow cell, are a Flongle Flow Cell, a MinION Flow Cell, and the PromethION Flow Cell, each also provided by Oxford Nanopore Technologies plc. The sequencing device 104 generates signals based on interactions between the sample 108 and the nanopores of the sequencing device 104, and provides the signals to the fusion finder computing system 106 for analysis.

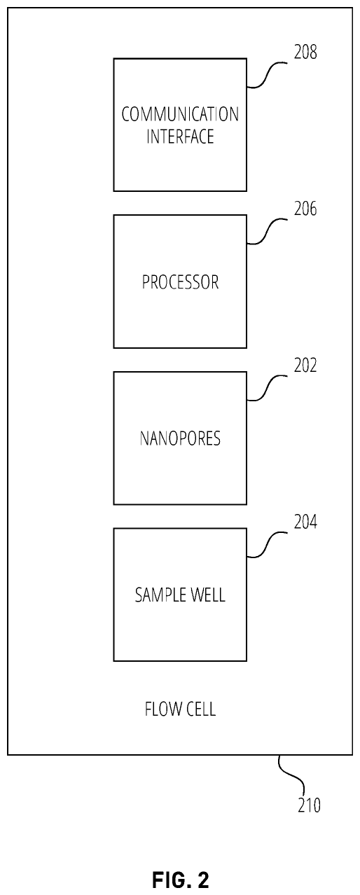

FIG. 2 is a schematic illustration of a non-limiting example embodiment of a flow cell according to various aspects of the present disclosure. The flow cell 210 is an example of a sequencing device 104, or a component thereof, illustrated in FIG. 1. As shown, the flow cell 210 includes a sample well 204, a plurality of nanopores 202, a processor 206, and a communication interface 208. The sample well 204 is configured to accept the sample 108 (e.g., to receive drops of sample 108 from a pipette) and to provide the sample 108 to the plurality of nanopores 202. The processor 206 is configured to control a voltage applied to the plurality of nanopores 202 and to read signals generated by the nanopores 202. In some embodiments, the processor 206 may also be configured to segment the signals generated by the nanopores 202 into a plurality of segmented events, each segmented event representing an interaction of a molecule with a nanopore 202 of the plurality of nanopores 202. In some embodiments, the processor 206 may be configured to perform base-calling (determining an identity of an amino acid represented by one or more segmented events). In some embodiments, the communication interface 208 is configured to transmit the signals detected by the processor 206, the segmented events, and/or the base-calling results to another device, such as the fusion finder computing system 106, using a wired or wireless network, a USB connection, or any other suitable communication technique. In some embodiments, the processor 206, communication interface 208, and potentially other components (such as a computer-readable medium) may be implemented on an ASIC or FPGA that is part of the flow cell 210.

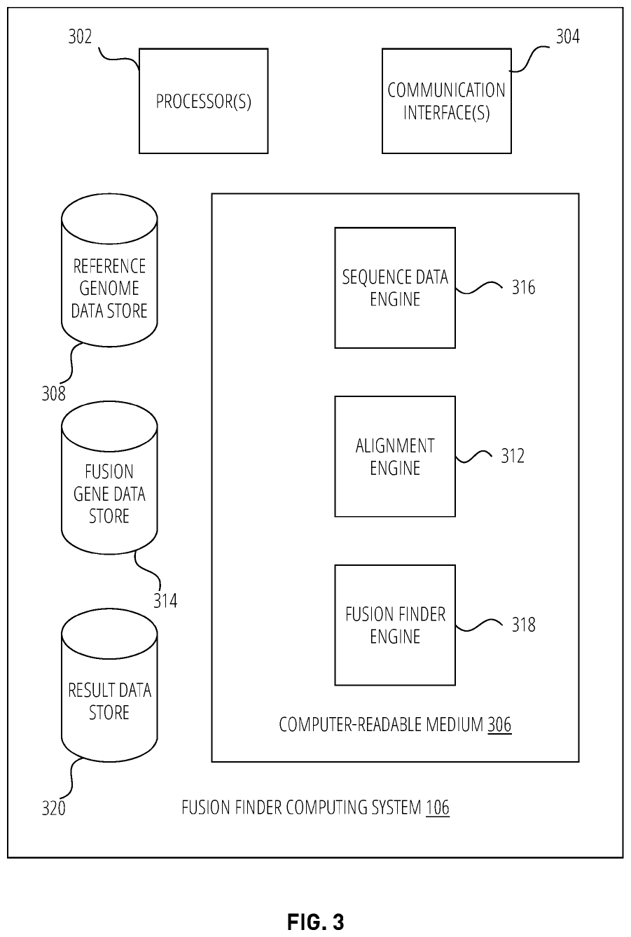

FIG. 3 is a block diagram that illustrates aspects of a non-limiting example embodiment of a fusion finder computing system according to various aspects of the present disclosure. The illustrated fusion finder computing system 106 may be implemented by any computing device or collection of computing devices, including but not limited to a desktop computing device, a laptop computing device, a mobile computing device, a server computing device, a computing device of a cloud computing system, and/or combinations thereof. In some embodiments, some portions of the fusion finder computing system 106 may be provided using a containerized computing platform (e.g., Docker, Kubernetes, etc.) in order to improve flexibility, scalability, and other performance aspects of the system. The fusion finder computing system 106 is configured to process sequencing information generated by sequencing devices to detect fusion genes, as described in further detail below.

As shown, the fusion finder computing system 106 includes one or more processors 302, one or more communication interfaces 304, a reference genome data store 308, a fusion gene data store 314, a result data store 320, and a computer-readable medium 306.

As used herein, “computer-readable medium” refers to a removable or nonremovable device that implements any technology capable of storing information in a volatile or non-volatile manner to be read by a processor of a computing device, including but not limited to: a hard drive; a flash memory; a solid state drive; random-access memory (RAM); read-only memory (ROM); a CD-ROM, a DVD, or other disk storage; a magnetic cassette; a magnetic tape; and a magnetic disk storage.

As used herein, “data store” refers to any suitable device configured to store data for access by a computing device. One example of a data store is a highly reliable, high-speed relational database management system (DBMS) executing on one or more computing devices and accessible over a high-speed network. Another example of a data store is a key-value store. However, any other suitable storage technique and/or device capable of quickly and reliably providing the stored data in response to queries may be used, and the computing device may be accessible locally instead of over a network, or may be provided as a cloud-based service. A data store may also include data stored in an organized manner on a computer-readable storage medium, such as a hard disk drive, a flash memory, RAM, ROM, or any other type of computer-readable storage medium. One of ordinary skill in the art will recognize that separate data stores described herein may be combined into a single data store, and/or a single data store described herein may be separated into multiple data stores, without departing from the scope of the present disclosure.

In some embodiments, the processors 302 may include any suitable type of general-purpose computer processor. In some embodiments, the processors 302 may include one or more special-purpose computer processors or AI accelerators optimized for specific computing tasks, including but not limited to graphical processing units (GPUs), vision processing units (VPTs), and tensor processing units (TPUs).

In some embodiments, the communication interfaces 304 include one or more hardware and or software interfaces suitable for providing communication links between components. The communication interfaces 304 may support one or more wired communication technologies (including but not limited to Ethernet, FireWire, and USB), one or more wireless communication technologies (including but not limited to Wi-Fi, WiMAX, Bluetooth, 2G, 3G, 4G, 5G, and LTE), and/or combinations thereof.

As shown, the computer-readable medium 306 has stored thereon logic that, in response to execution by the one or more processors 302, cause the fusion finder computing system 106 to provide a sequence data engine 316, an alignment engine 312, and a fusion finder engine 318.

As used herein, “engine” refers to logic embodied in hardware or software instructions, which can be written in one or more programming languages, including but not limited to C, C++, C#, COBOL, JAVA™, PHP, Perl, HTML, CSS, Javascript, VBScript, ASPX, Go, and Python. An engine may be compiled into executable programs or written in interpreted programming languages. Software engines may be callable from other engines or from themselves. Generally, the engines described herein refer to logical modules that can be merged with other engines, or can be divided into sub-engines. The engines can be implemented by logic (e.g., computer-executable instructions) stored in any type of computer-readable medium or computer storage device and be stored on and executed by one or more general purpose computers, thus creating a special purpose computer configured to provide the engine or the functionality thereof. The engines can be implemented by logic programmed into an application-specific integrated circuit (ASIC), a field-programmable gate array (FPGA), or another hardware device. The engines can be implemented in containers and executed in a cloud or non-cloud environment by a container management and orchestration system including but not limited to Docker or Kubernetes.

In some embodiments, the sequence data engine 316 is configured to determine a plurality of read sequences based on sequencing data generated by a sequencing device 104. In some embodiments, the alignment engine 312 is configured to determine alignments of the read sequences to one or more reference genomes from the reference genome data store 308. In some embodiments, the fusion finder engine 318 is configured to determine whether the alignments indicate the presence of one or more fusion genes from the fusion gene data store 314, and if so, to store indications of the presence of the one or more fusion genes in the result data store 320.

Further description of the configuration of each of these components is provided below.

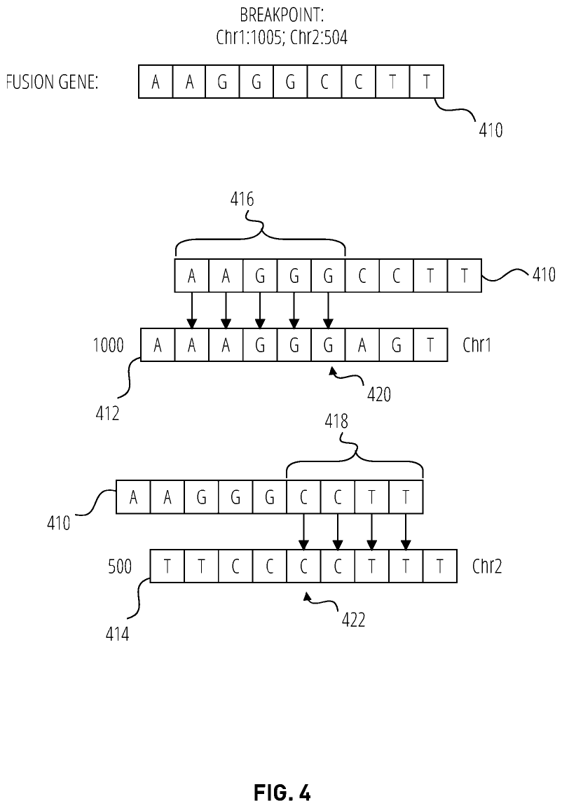

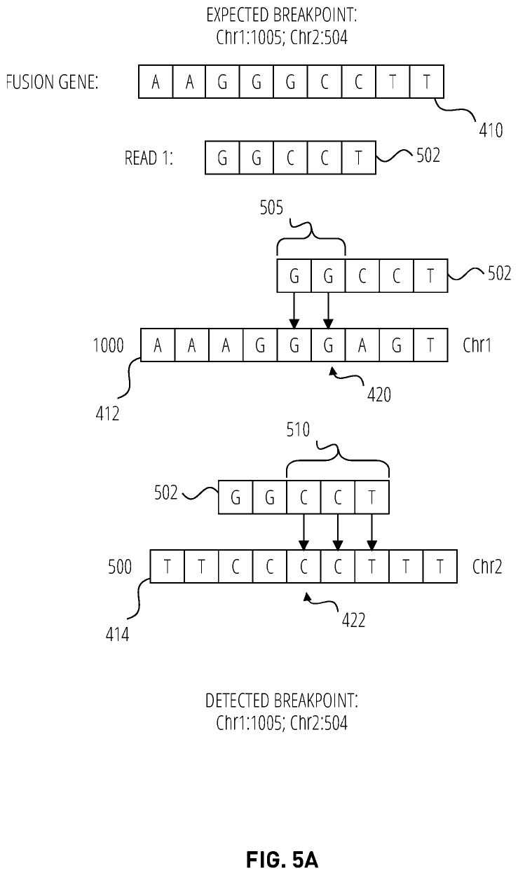

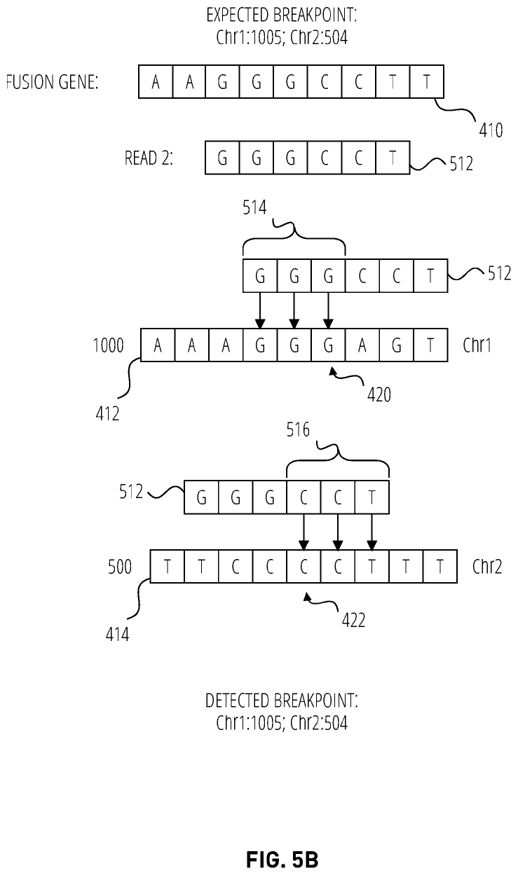

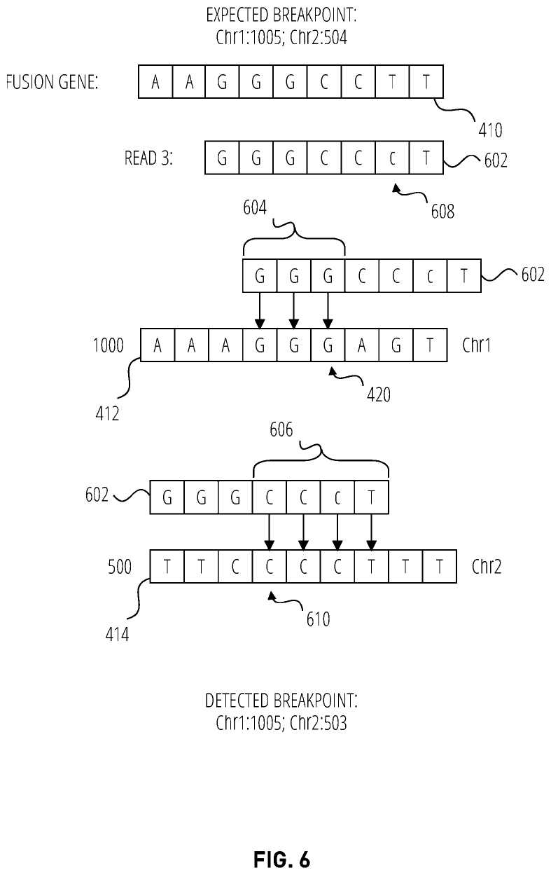

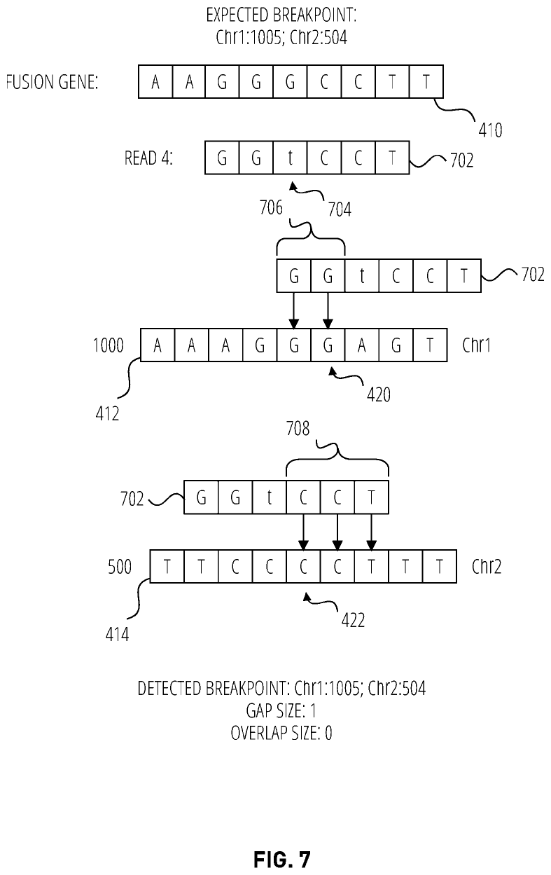

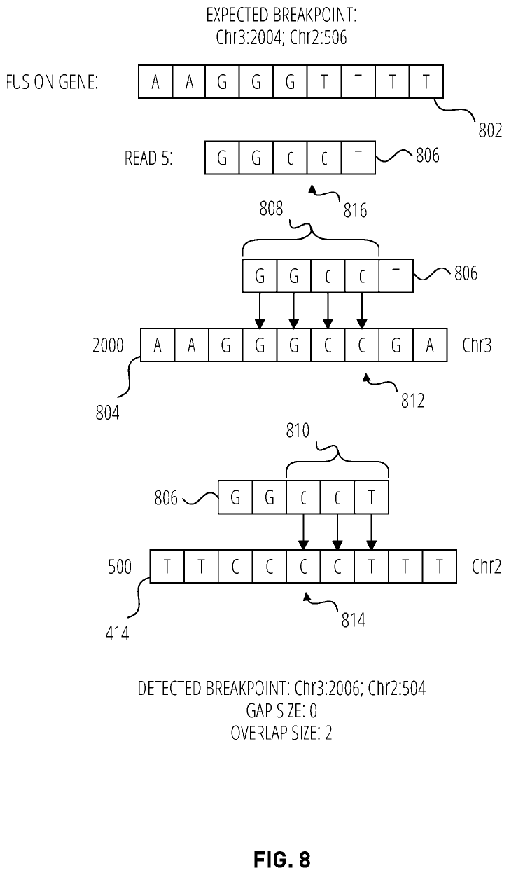

FIG. 4 is a schematic illustration of a fictional fusion gene according to various aspects of the present disclosure. The fictional sequences provided in FIG. 4 (as well as FIG. 5A, FIG. 5B, FIG. 6, FIG. 7, and FIG. 8) are provided for the purposes of illustrating the techniques described herein, and are not intended to represent any actual fusion gene sequence or chromosomal sequence from an actual reference genome. In actual embodiments, the fusion gene sequence, the chromosome sequences, and/or the read sequences may be considerably longer than those illustrated herein, including but not limited to reads hundreds of bases long (for second generation sequencing technologies), thousands of bases long (for third generation sequencing technologies), or other lengths.

The illustrated fusion gene 410 results from a translocation between a first chromosome 412 and a second chromosome 414. The first chromosome 412 is illustrated beginning at location 1000, and is labeled as “Chr1”. The second chromosome 414 is illustrated beginning at location 500, and is labeled as “Chr2”.

A first portion 416 of the fusion gene 410 comes from the first chromosome 412, and a second portion 418 of the fusion gene 410 comes from the second chromosome 414, as indicated by the arrows between the fusion gene 410 and the respective chromosomes. The breakpoint that describes the fusion gene 410 includes a first breakpoint coordinate 420 (the location of the last base taken from the first chromosome 412) and a second breakpoint coordinate 422 (the location of the first base taken from the second chromosome 414). Accordingly, since the last base taken from the first chromosome 412 is from position 1005, and the first base taken from the second chromosome 414 is from position 504, the breakpoint location for the fusion gene 410 is denoted as “Chr1:1005; Chr2:504.”

Though a fusion gene 410 resulting from a translocation is illustrated and described herein for the sake of simplicity, one will note that fusion genes formed in other ways, such as interstitial deletion or chromosomal inversion wherein both portions of the fusion gene come from the same chromosome, may also be processed using the techniques disclosed herein.

FIG. 5A and FIG. 5B illustrate a first read sequence and a second read sequence that indicate the presence of the fusion gene illustrated in FIG. 4 without any errors. In FIG. 5A, the first read sequence 502 is illustrated. A first portion 505 of the first read sequence 502 is aligned to the first chromosome 412, and a second portion 510 of the first read sequence 502 is aligned to the second chromosome 414. The last base from the first chromosome 412 aligned to the first portion 505 was at the first breakpoint coordinate 420 (Chr1:1005), and the first base from the second chromosome 414 aligned to the second portion 510 was at the second breakpoint coordinate 422 (Chr2:504). As such, the first read sequence 502 provides an exact match to the breakpoint location expected for the fusion gene 410.

In FIG. 5B, the second read sequence 512 is illustrated. Again, a first portion 514 of the second read sequence 512 is aligned to the first chromosome 412, and a second portion 516 of the second read sequence 512 is aligned to the second chromosome 414. The last base from the first chromosome 412 aligned to the first portion 514 was at the first breakpoint coordinate 420 (Chr1:1005), and the first base from the second chromosome 414 aligned to the second portion 516 was at the second breakpoint coordinate 422 (Chr2:504). As such, the second read sequence 512 also provides an exact match to the breakpoint location expected for the fusion gene, and further supports its presence in the data.

FIG. 6 illustrates a third read sequence that should be considered as indicating the presence of the fusion gene illustrated in FIG. 4, but that includes a minor sequencing error that leads to a change in the detected breakpoint. In the third read sequence 602, an insertion error 608 is present, such that an extra base is present in the third read sequence 602 that is not present in the fusion gene 410. It is assumed that the insertion error 608 does not reflect an actual base present in the molecule that was sequenced, but is instead an artifact introduced either during generation of the sequencing data or during base calling.

Once the third read sequence 602 is aligned to the fusion gene 410, the effect of the insertion error 608 is apparent. A first portion 604 of the third read sequence 602 still aligns to the first chromosome 412 and a second portion 606 of the third read sequence 602 still aligns to the second chromosome 414. However, the presence of the insertion error 608 changes the breakpoint location associated with the third read sequence 602: as shown, the last base from the first chromosome 412 aligned to the first portion 604 is still at the first breakpoint coordinate 420 (Chr1:1005), but the first base from the second chromosome 414 aligned to the second portion 606 is now at a third breakpoint coordinate 610 (Chr2:503). This means that the breakpoint location indicated by the third read sequence 602 is “Chr1:1005; Chr2:503,” which does not exactly match the expected breakpoint of “Chr1:1005; Chr2:504.”

Previous techniques for detecting fusion genes such as LongGF would not consider the third read sequence 602 to indicate the presence of the fusion gene 410 because of the mismatch between the detected breakpoint and the expected breakpoint. This is particularly problematic when using next-generation sequencing or other sequencing technologies that are particularly susceptible to generating these types of sequencing errors.

FIG. 7 illustrates a fourth read sequence that should be considered as indicating the presence of the fusion gene illustrated in FIG. 4, but that includes a minor sequencing error that leads to a gap in coverage of the fourth read sequence. In the fourth read sequence 702, another insertion error 704 is present, such that an extra base is present in the fourth read sequence 702 that is not present in the fusion gene 410. Again, it is assumed that the insertion error 704 does not reflect an actual base present in the molecule that was sequenced, but is instead an artifact introduced either during generation of the sequencing data or during base calling.

After alignment, a first portion 706 of the fourth read sequence 702 is aligned to the first chromosome 412, and a second portion 708 of the fourth read sequence 702 is aligned to the second chromosome 414. The last base from the first chromosome 412 aligned to the first portion 706 is at the first breakpoint coordinate 420 (Chr1:1005) and the first base from the second chromosome 414 aligned to the second portion 708 is at the second breakpoint coordinate 422 (Chr2:504), and so the detected breakpoint location matches the expected breakpoint location. However, the base represented by the insertion error 704 is not present in either the first portion 706 or the second portion 708. Bases such as these that are not present in the portion of the read sequence aligned to the first chromosome 412 or the second chromosome 414 are referred to herein as a “gap,” and the number of such bases is referred to herein as a “gap size.” As shown in FIG. 7, because the insertion error 704 causes a single base to not be present in the first portion 706 or the second portion 708, the gap size value for this example is one.

Previous techniques for detecting fusion genes such as LongGF may also not consider the fourth read sequence 702 to indicate the presence of the fusion gene 410 because the entire fourth read sequence 702 is not aligned to either the first chromosome 412 or the second chromosome 414. Since such gaps can be introduced by insertion errors, and insertion errors are common when using the sequencing technologies described herein, the failure of previous techniques to accommodate for non-zero gap size values causes previous techniques to be unsuccessful in detecting many fusion genes.

FIG. 8 illustrates another potential issue that may arise in attempting to detect fusion genes using read sequences. In FIG. 8, a second fusion gene 802 different from the fusion gene 410 illustrated in the other figures, which is a translocation between a third chromosome 804 and the second chromosome 414. This second fusion gene 802 is also fictional and is provided for the sake of discussion. As illustrated, the second fusion gene 802 has an expected breakpoint location of “Chr3:2004; Chr2:506.” The fifth read sequence 806 includes an insertion error 816 of two bases, but would otherwise align correctly and indicate the presence of the second fusion gene 802.

After alignment, a first portion 808 of the fifth read sequence 806 has been aligned to the third chromosome 804, and a second portion 810 of the fifth read sequence 806 has been aligned to the second chromosome 414. The last base from the third chromosome 804 aligned to the first portion 808 is at a third breakpoint coordinate 812 (Chr3:2006), and the first base from the second chromosome 414 aligned to the second portion 810 is at a fourth breakpoint coordinate 814 (Chr2:504). The detected breakpoint location is therefore “Chr3:2006; Chr2:504,” which does not exactly match the expected breakpoint location. One will also note that the bases of the insertion error 816 are part of both the first portion 808 and the second portion 810. Bases such as these that are present in both the portion of the read sequence aligned to the third chromosome 804 and the portion of the read sequence aligned to the second chromosome 414 are referred to herein as an “overlap”,” and the number of such bases is referred to herein as an “overlap size.” As shown in FIG. 8, because two bases are present in both the first portion 808 and the second portion 810, the overlap size value for this example is two.

Previous techniques for detecting fusion genes such as LongGF may not consider the fifth read sequence 806 to indicate the presence of the second fusion gene 802 due to the non-zero overlap size value. Since non-zero overlap size values may be introduced by insertion errors common to the sequencing technologies described herein, the failure of previous techniques to accommodate for non-zero overlap size values causes previous techniques to be unsuccessful in detecting many fusion genes.

What is desired are computing techniques that can successfully and efficiently detect fusion genes in sequencing data, even if breakpoint mismatches, gaps, and/or overlaps are present due to minor sequencing errors or for other reasons.

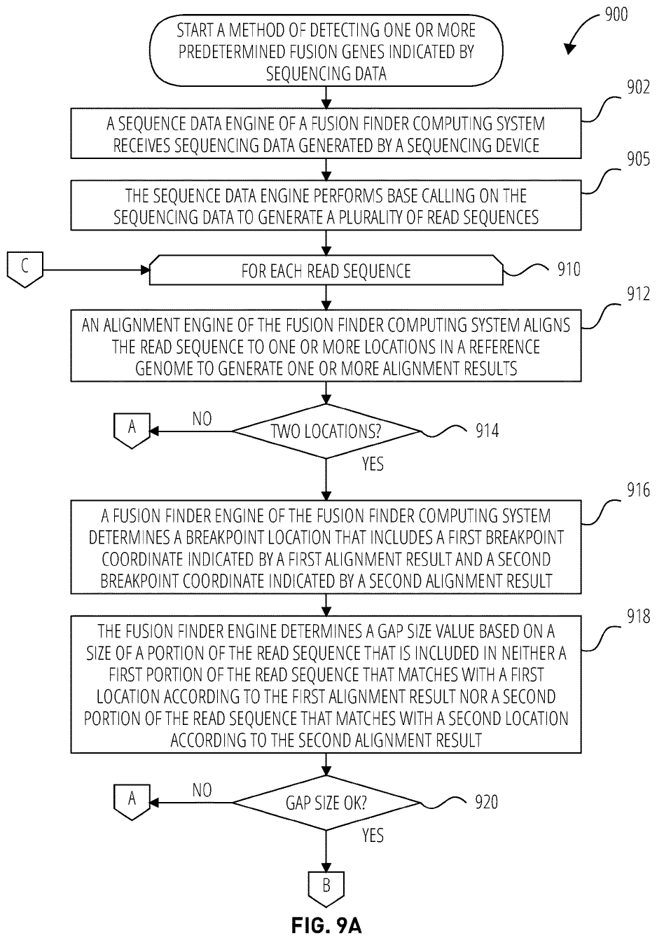

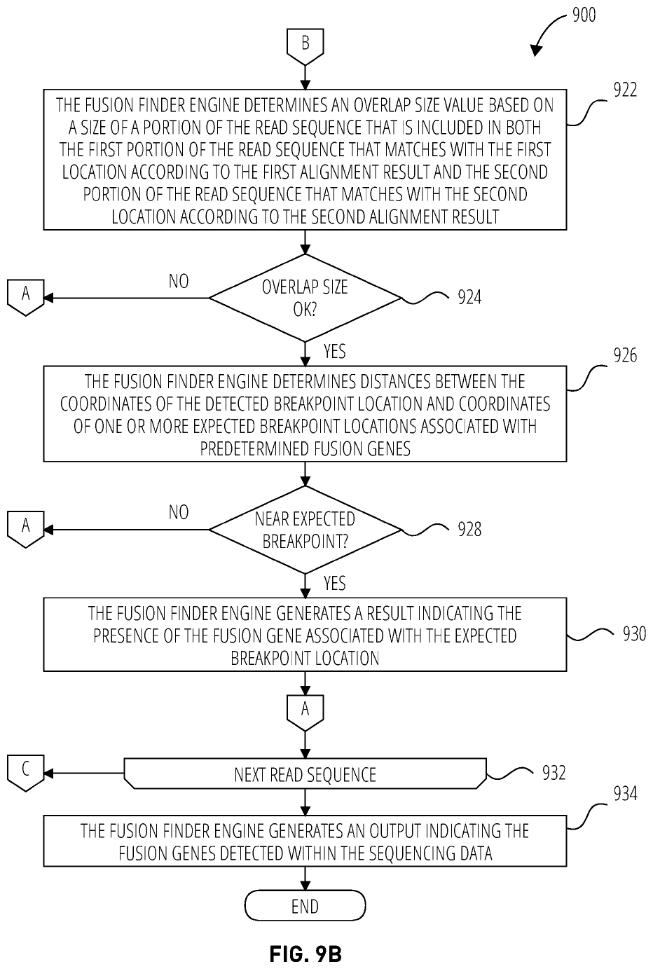

FIG. 9A and FIG. 9B are a flowchart that illustrates a non-limiting example embodiment of a method of detecting one or more predetermined fusion genes indicated by sequencing data, according to various aspects of the present disclosure. In the method 900, alternate alignments for each read are examined and reads that map to coordinates spanning a set of expected breakpoint locations are identified. This strategy is different from previous techniques such as LongGF, at least because the method 900 allows for mismatches in the alignment near the breakpoint location. By compensating for these mismatches, the method 900 is capable of detecting fusion genes not detected by LongGF.

From a start block, the method 900 proceeds to block 902, where a sequence data engine 316 of a fusion finder computing system 106 receives sequencing data generated by a sequencing device 104. In some embodiments, the sequence data engine 316 may receive signals from the sequencing device 104 while the sample 108 is being sequenced, referred to as “streaming” the signals from the sequencing device 104. In some embodiments, the sequence data engine 316 may receive a file (e.g., a fast5 file, or another suitable format) generated by the sequencing device 104 after sequencing has been completed.

At block 905, the sequence data engine 316 performs base calling on the sequencing data to generate a plurality of read sequences. The sequence data engine 316 may use any suitable technique for performing base calling on the sequencing data, including but not limited to using a Guppy base caller provided by Oxford Nanopore Technologies, a Scrappie base caller (also provided by Oxford Nanopore Technologies, or any other suitable base caller. One technical advantage of using the Guppy base caller (or similar base callers) is that Guppy provides the ability to use GPUs to improve processing times.

Though the method 900 describes the sequence data engine 316 as receiving sequencing data and performing base calling to generate the plurality of read sequences, in some embodiments, the sequence data engine 316 may instead receive the plurality of read sequences themselves from another device or system, or may retrieve the plurality of read sequences from a data store.

The method 900 then proceeds to a for-loop defined between for-loop start block 910 and for-loop end block 932, wherein each read sequence of the plurality of read sequences is processed to determine whether it indicates presence of one of the predetermined fusion genes.

From the for-loop start block 910, the method 900 proceeds to block 912, where an alignment engine 312 of the fusion finder computing system 106 aligns the read sequence to one or more locations in a reference genome to generate one or more alignment results. The alignment engine 312 may retrieve the reference genome from the reference genome data store 308. In some embodiments, the reference genome may be from a given organism, such as a human genome. In some embodiments, the reference genome may include genomic information from multiple organisms, such as information from a human genome and also information from a genome of a pathogen, in order to detect chimeric genes having multiple biological sources. In some embodiments, the reference genome may be limited to a portion of interest of the genome of the organism(s), in order to improve the efficiency of the alignment. Any alignment tool, including but not limited to the open source minimap2 tool (Li, H., “Minimap2: pairwise alignment for nucleotide sequences,” Bioinformatics, 2018 Sep. 15; 34 (18): 3094-3100, incorporated by reference herein in its entirety for all purposes), Samtools (available from Genome Research Limited), and/or other tools may be used by the alignment engine 312 to align the read sequence to the one or more locations in the reference genome.

Depending on the content of the read sequence, the alignment engine 312 may align the read sequence to no locations in the reference genome (if an alignment cannot be found), one location in the reference genome (if only a single alignment is found), or more than one location in the reference genome (if multiple potential alignments are found). A read sequence that aligns to a fusion gene would match to one location before the fusion gene breakpoint, and to another location after the fusion gene breakpoint. Therefore, read sequences that match to two (or potentially more) locations within the reference genome are of interest to the method 900.

Accordingly, at decision block 914, a determination is made regarding whether the alignment engine 312 aligned the read sequence to two locations in the reference genome. The method 900 is illustrated as requiring exactly two locations for the ease of illustration and discussion. However, this example should not be seen as limiting. In some embodiments, if a read sequence aligns to more than two locations in the reference genome, the method 900 may consider each pair of locations within the reference genome to which the read sequence aligns separately.

If it is determined that the read sequence is not aligned to two locations in the reference genome, then the result of decision block 914 is NO, and the method 900 advances through a continuation terminal (“terminal A”) to the end of the for-loop. Otherwise, if it is determined that the read sequence is aligned to two locations in the reference genome, then the result of decision block 914 is YES, and the method 900 advances to block 916. At block 916, a fusion finder engine 318 of the fusion finder computing system 106 determines a breakpoint location that includes a first breakpoint coordinate indicated by a first alignment result and a second breakpoint coordinate indicated by a second alignment result. As illustrated above, the first alignment result aligns a first portion of the read sequence to a first chromosome, and the second alignment result aligns a second portion of the read sequence to a second chromosome. The first breakpoint coordinate is a coordinate of a last base of the first chromosome aligned to the first portion of the read sequence, and the second breakpoint coordinate is a coordinate of a first base of the second chromosome aligned to the second portion of the read sequence.

At block 918, the fusion finder engine 318 determines a gap size value based on a size of a portion of the read sequence that is included in neither a first portion of the read sequence that matches with a first location according to the first alignment result nor a second portion of the read sequence that matches with a second location according to the second alignment result. If the entire read sequence is included in either the first portion or the second portion, then the gap size value will be zero. As illustrated in FIG. 7, any portion of the read sequence that is not covered by either the first portion or the second portion will be considered the gap, and the number of bases in the gap is determined to be the gap size value.

The method 900 then advances to a decision block 920, where the fusion finder engine 318 compares the gap size value to a gap size value threshold to determine whether it is worth continuing to process the read sequence, or whether the size of the gap is too large for the read sequence to be a likely indicator of the presence of a fusion gene. Any suitable gap size value threshold may be used, and may be configured by an operator in order to provide desired results. One non-limiting example of a suitable gap size value threshold is two, though in other embodiments, other gap size value thresholds, such as gap size value thresholds within a range of 2-10 may be used.

If the gap size value does not satisfy the gap size value threshold, then the result of decision block 920 is NO, and the method 900 advances through terminal A to the end of the for-loop, skipping further processing of the read sequence. Otherwise, if the gap size value does satisfy the gap size threshold, then the result of decision block 920 is YES, and the method 900 advances to a continuation terminal (“terminal B”).

From terminal B (FIG. 9B), the method 900 advances to block 922, where the fusion finder engine 318 determines an overlap size value based on a size of a portion of the read sequence that is included in both the first portion of the read sequence that matches with the first location according to the first alignment result and the second portion of the read sequence that matches with the second location according to the second alignment result. As illustrated in FIG. 8, any portion of the read sequence that is covered by both the first portion and the second portion will be considered the overlap, and the number of bases in the overlap is determined to be the overlap size value.

The method 900 then advances to a decision block 924, where the fusion finder engine 318 compares the overlap size value to an overlap size value threshold to determine whether it is worth continuing to process the read sequence, or whether the size of the overlap is too large for the read sequence to be a likely indicator of the presence of a fusion gene. Any suitable overlap size value threshold may be used, and may be configured by an operator in order to provide the desired results. One non-limiting example of a suitable overlap size value threshold is two, though in other embodiments, other overlap size value thresholds, such as overlap size value thresholds within a range of 2-10 may be used.

If the overlap size value does not satisfy the overlap size value threshold, then the result of decision block 924 is NO, and the method 900 advances through terminal A to the end of the for-loop, skipping further processing of the read sequence. Otherwise, if the overlap size value does satisfy the overlap size value threshold, then the result of decision block 924 is YES, and the method 900 advances to block 926.

At block 926, the fusion finder engine 318 determines distances between the coordinates of the detected breakpoint location and coordinates of one or more expected breakpoint locations associated with predetermined fusion genes. In some embodiments, the fusion finder engine 318 may retrieve a panel of expected breakpoint locations from the fusion gene data store 314. The fusion finder engine 318 may determine differences between each coordinate of each expected breakpoint location and corresponding coordinates of the detected breakpoint location of the alignments for the read sequence.

The distance between the detected breakpoint location and the expected breakpoint location may then be determined in any suitable way, such as by adding the differences together, taking the largest distance, or in any other suitable way. For example, in FIG. 6, the expected breakpoint location has breakpoint coordinates of Chr:1005; Chr2:504, while the detected breakpoint location has breakpoint coordinates of Chr:1005; Chr2:503. The first breakpoint coordinates match, while there is a difference of one between the second breakpoint coordinates. If the two distances are added, the distance between this expected breakpoint location and the detected breakpoint location would be one. Likewise, in FIG. 8, the expected breakpoint location has breakpoint coordinates of Chr3:2004; Chr2:506, while the detected breakpoint location has breakpoint coordinates of Chr3:2006; Chr2:504. The first breakpoint coordinates have a difference of two, and the second breakpoint coordinates have a distance of two. If the two distances are added, the distance between this expected breakpoint location and the detected breakpoint location would be four.

The method 900 then advances to a decision block 928, where the fusion finder engine 318 determines whether the detected breakpoint location is near an expected breakpoint location. To determine whether the detected breakpoint location is near an expected breakpoint location, the distance between the detected breakpoint location and the expected breakpoint location determined at block 926 may be compared to a distance threshold, and if the distance satisfies the distance threshold, then the detected breakpoint location may be determined to be near the associated expected breakpoint location. Any suitable distance threshold may be used, and may be configured by an operator in order to provide the desired results. One non-limiting example of a suitable distance threshold is two, though in other embodiments, other distance thresholds, such as distance thresholds within a range of 2-10 may be used.

In some embodiments, a static distance threshold may be used, while in other embodiments, the distance threshold may be adjusted based on the gap size value and/or the overlap size value. For example, as illustrated in FIG. 8, the insertion error 816 leads to both a non-zero distance value and a non-zero overlap size value. In some embodiments, the non-zero overlap size value may be used to adjust the distance threshold since the same insertion error 816 will contribute to both the overlap size and the distance.

If the breakpoint location is not near an expected breakpoint location, then the result of decision block 928 is NO, and the method 900 advances through terminal A to the end of the for-loop. Otherwise, if the breakpoint location is near an expected breakpoint location, then the result of decision block 928 is YES, and the method 900 advances to block 930, where the fusion finder engine 318 generates a result indicating the presence of the fusion gene associated with the expected breakpoint location. In some embodiments, the result may include the read sequence and the alignments of the read sequence that are associated with the fusion gene. In some embodiments, the fusion finder engine 318 may store the result in the result data store 320.

The method 900 then proceeds through terminal A to the for-loop end block 932. From for-loop end block 932, if further read sequences remain to be processed, then the method 900 proceeds to a continuation terminal (“terminal C”), and from terminal C back to the for-loop start block 910 to process the next read sequence. Otherwise, if all of the read sequences have been processed, then the method 900 advances from for-loop end block 932 to block 934.

At block 934, the fusion finder engine 318 generates an output indicating the fusion genes detected within the sequencing data. The fusion finder engine 318 may retrieve the results from the result data store 320, and may generate a tabular output, a text file output, a visualization, or any other suitable format of output. In some embodiments, the fusion finder engine 318 may use a tool such as the Integrative Genomics Viewer (IGV) to show how each of the read sequences that were found to be indicative of the fusion gene are aligned to the fusion gene, or summaries thereof.

The method 900 then proceeds to an end block and terminates.

As illustrated, the method 900 is organized with an if-then-else logical structure that causes the method 900 to first check for a valid gap size (and to skip further processing of a read sequence if the gap size is not valid); then to check for a valid overlap size (and to skip further processing of the read sequence if the overlap size is not valid); and then to compare the detected breakpoint location to a panel of expected breakpoint locations. Since the comparison of the detected breakpoint location to the panel of expected breakpoint locations is the most computationally expensive task performed by the fusion finder engine 318, skipping further processing if the gap size or overlap size is not valid allows execution of this computationally expensive task to be minimized, thereby improving processing time even for large panels of expected breakpoint locations. However, this logic should be viewed as an example only, and should not be seen as limiting. In some embodiments, the overlap size may be validated prior to the gap size. In some embodiments, the further processing may not be skipped, and the overlap size, gap size, and distances may all be checked for every read sequence.

Further, the illustrated method 900 assumes the presence of a panel of expected breakpoint locations. However, in some embodiments, similar techniques may be used to detect fusion genes in the absence of a panel of expected breakpoints. For example, each read sequence of the plurality of read sequences may be processed as described from block 912 to decision block 924 in order to determine a breakpoint location, a gap size value and an overlap size value for read sequences that align to two locations. If the gap size value is less than a gap size value threshold and the overlap size value is less than an overlap size value threshold, the breakpoint location may be added to a set of candidate breakpoint locations instead of comparing the breakpoint location to the expected breakpoint locations as described in block 926. Once a set of candidate breakpoint locations is determined, a similar breakpoint set may be determined by finding candidate breakpoints that are within a threshold distance of each other (e.g., within a distance of each other such as the distance threshold discussed above, as opposed to within the threshold distance from an expected breakpoint). If the similar breakpoint set has enough candidate breakpoints added to it (that is, the size of the similar breakpoint set is larger than a threshold size), it is an indication that the read sequences related to the similar breakpoint set support the presence of a fusion gene near the candidate breakpoint locations. As such, if the size of the similar breakpoint set is larger than the threshold, an indication may be generated of a detected fusion gene associated with the similar breakpoint set.

### Example Protocol

The method 900 for detecting fusion genes in sequencing data may be incorporated into a testing protocol in which a sample 108 obtained from a subject 102 is collected, sequenced, and tested for fusion genes that may indicate a presence or absence of a condition. One non-limiting example of such a protocol was developed to validate the effectiveness of the method 900, and can be used for testing actual samples. The tested protocol is an amplification-free CRISPR-Cas9 targeted enrichment sequencing protocol using Nanopore MinION flow cells and Flongles to detect fusion genes relevant to the diagnosis and classification of chronic myelogenous leukemia (CML) and acute myeloid leukemias (AMLs), but could also be used to detect other fusion genes. Flongle, from Oxford Nanopore Technologies, is an adaptor for the MinION device that provides cost-effective (˜USD $90 per MinION flow cell) real-time sequencing for smaller assays of limited target genes. The assays disclosed herein were designed to capture various breakpoints of CML and APL, as well as fusion genes resulting from inv(16) (CBFB::MYH11) and t(4;11) (KMT2A::AFF1). Simultaneous interrogation of these targets helps enable rapid characterization of AMLs in a single assay combining data that previously required multiple different techniques and provides relevant information promptly. Using the optimized assay and the method 900, fusion breakpoints were detected and confirmed in 80% of tested specimens in under 3 hours total time, including both sequencing and data analysis.

The example assay was optimized using six cell lines: three with the BCR::ABL1 fusion (K562, KU812, and KCL22), and NB4, MV4;11 and ME-1 that bear the PML::RARA, KMT2A::AFF1, and CBFB::MYH11 fusion genes, respectively. Residual mononuclear cells from primary specimens (six specimens from 5 patients with CML, six specimens from 5 patients with suspected APL, and two acute myeloid leukemia, not acute promyelocytic leukemia) were isolated using Ficoll reagent (Millipore-Sigma) and banked in liquid nitrogen until the time of the experiment. All specimens had been originally tested in a CLIA-certified laboratory according to standard clinical protocols. IRB coverage was obtained for the use of residual laboratory samples. Patient samples were de-identified to the Nanopore testing lab, and cytogenic or molecular results were confirmed after nanopore results were rendered. Characteristics and demographics of specimens and patients are listed in Table 1:

TABLE 1

| Patient | | | | Specimen | Disease | |
|---|---|---|---|---|---|---|
| code | Age | Gender | Diagnosis | Source | Burden | Cytogenics/Molecular |
| CML1 | 62 | Male | CML- | BM | 2% | 46, XY, t(5; 12)(q33; q15), t(9; 22) |
| | | | AP Rlps | | blasts | (q34; q11.2)[2] / 47, sl, +8[18] |
| CML2 | 45 | Female | CML-BC | PB | NA | Outside cytogenetics confirmed |
| | | | | | | t(9; 22) but only in 3/21 |
| | | | | | | karyotypes. Full karyotype not |
| | | | | | | available. |
| CML3 | 56 | Male | CML-AP | PB | NA | Patient with long standing p190 |
| | | | | | | CML. At this timepoint sample |
| | | | | | | presents MDS/MPN with low |
| | | | | | | level BCR::ABL1 by PCR. |
| CML4 | 21 | Male | CML-CP | PB | 3% | 46, XX, t(9; 22)(q34; q11.2)[20] |
| | | | | | blasts | |
| CML5 | 62 | Male | CML- | PB | 3% | 46, XY, t(5; 12)(q33; q15), t(9; 22) |
| | | | AP Rlps | | blasts | (q34; q11.2)[2] / 47, sl, +8[18] |
| CML6 | 70 | Female | CML-BC | PB | 16% | 46, XX, t(9; 22)(q34: q11.2)[4]; |
| | | | | | blasts | Confirmed by molecular studies. |
| AML1 | 39 | Male | AML | PB | 90% | 46, XY, t(9; 11)(q21; q23)[20] |
| | | | | | blasts | |
| AML2 | 40 | Female | AML | PB | 20% | 47, XX +8, inv(16)(p13.1q22)[17] / |
| | | | | | blasts | 46, XX[3] |
| APL1 | 83 | Male | APL | PB | <1% | Patient with collision multiple |
| | | | | | blasts | myeloma and APL in BM with |
| | | | | | | 30% blasts, not available for |
| | | | | | | analysis. No circulating APL in |
| | | | | | | PB |
| APL2 | 34 | Female | APL | BM | 80% | 46, XX, der(2)t(2; 17)(q33; q21) |
| | | | | | blasts | t(15; 17)(q22; q21), der(15) |
| | | | | | | t(15; 17), der(17)t(2; 17) [3] / |
| | | | | | | 47, sl +8[3]/48, sdl, +8[12] / |
| | | | | | | 46, XX[2] |
| APL3 | 23 | Male | APL | PB | 75% | NA |
| | | | | | blasts | |
| APL4 | 39 | Male | APL | BM | 83% | AML with isochromosome 17q |
| | | | | | blasts | |
| APL5 | 39 | Male | APL | PB | 79% | AML with isochromosome 17q |
| | | | | | blasts | |
| APL6 | 43 | Male | APL | BM | 22% | 46, XY, t(1; 7)(q21; q21), |
| | | | | | blasts in | t(4; 10)(q21; q25), add(8)(p23), |
| | | | | | a <5% | del(9)(p22), t(11; 17)(q23; q25) |
| | | | | | marrow | [4] / 46, XY[16] from 3 months |
| | | | | | | prior |

For the cell lines and 11/14 patient specimens, the DNA was extracted with PureGene (Qiagen, Germantown, MD, USA) following the standard protocol. Special caution, including the use of wide-bore pipette tips and moderate centrifuge spin velocity, was exercised to minimize fragmenting DNA strands. Two DNA specimens were extracted with AllPrep DNA/RNA Kit (Qiagen, Germantown, MD, USA) and one with QiAgen X-tractor with Reagent Pack DX (Qiagen, Germantown, MD, USA). cRNA guides were designed to direct Cas9 to cut on the genomic proximity of each region involved in the translocations studied. When the target region was large, guides were tiled across the region to maximize coverage. Guides were designed to capture PML::RARA, BCR::ABL1 p210, KMT2A::AFF1, and CBFB::MYH11, including different fusion isoforms. Guides were designed using Chopchop (available at https://chopchop.cbu.uib.no/) with the CRISPR-Cas9 and nanopore enrichment settings described in Sala-Torra et al., “TTMV-RARA fusion as a recurrent cause of AML with APL characteristics.” Blood Adv 2022; 6 (12): 3590-3592. doi: https://doi.org/10.1182/bloodadvances.2022007256, the entire disclosure of which is incorporated herein by reference.

The example protocol used five micrograms of DNA as input for each cell line and 2 to 5 micrograms for primary specimens. The average DNA integrity number (DIN) was 9.2 (range: 7.5-9.8). Details of the library prep were published in Sala-Torra et al., cited above. Briefly, enrichment of target regions was obtained using the protocol described in Gilpatrick, T. et al., “Targeted nanopore sequencing with Cas9-guided adapter ligation.” Nat. Biotechnol. 2020, 38 (4): 433-08. https://doi.org/10.1038/s41587-020-0407-5, the entire disclosure of which incorporated herein by reference. The different guides used in the assay were pooled in equimolar amounts of each guide. Through an initial dephosphorylation step, the 5′ ends of the DNA become inaccessible to adapter ligation. Double-stranded DNA breaks that excise the region of interest are generated with the directional, target-specific RNA guides complexed with tracrRNA and Cas9 enzyme. The Cas9 complex remains bound to the 5′ end of the guide, and the resulting new DNA ends contain a phosphorylated 5′ end that is available for dA tailing and adapter ligation. All libraries generated in this manner were run on a MinION nanopore sequencer (Oxford Nanopore Technologies, Oxford, UK) using MinION flow cells version 9.4 or Flongles. Modifications for libraries sequenced on the Flongle were only at the library loading step, in which the amount of Sequencing Buffer and library beads (both SQK-LSK 109, ONT) are reduced from 35 to 13 and 25.5 to 7.5 UL respectively, and 0.5 UL of SQT is added. QC parameters tracked for each run are listed in Table 2.

TABLE 2

| | | | GB | | | N50 | Median | | % |
|---|---|---|---|---|---|---|---|---|---|
| | | | of | Mean | All | A11 | Read | Median | Reads |
| Sample | DIN | Device | Data | Coverage | Reads | Reads | Length | PHRED | Aligned |
| K562 | 9.8 | Flongle | 0.12 | 0.04 | 35943 | 5980 | 2200 | 9.52 | 87.57 |
| KU812 | 8.7 | Flowcell | 0.36 | 0.11 | 86579 | 9690 | 1820 | 12.76 | 96.29 |
| KCL22 | 9.7 | Flowcell | 0.69 | 0.21 | 50439 | 32200 | 6290 | 12.26 | 91.86 |
| NB4 | 9.1 | Flongle | 0.07 | 0.02 | 23122 | 5890 | 1450 | 9.13 | 88.86 |
| MV4; 11 | 8.6 | Flowcell | 0.28 | 0.08 | 40000 | 27100 | 1890 | 11.98 | 89.34 |
| ME1 | 8.2 | Flowcell | 0.91 | 0.28 | 433846 | 16500 | 872 | 11.17 | 84.69 |
| CML1 | 9.8 | Flowcell | 0.92 | 0.28 | 32723 | 24600 | 9600 | 12.31 | 91.74 |
| CML2 | 9.6 | Flowcell | 0.04 | 0.01 | 4026 | 22400 | 6520 | 11.15 | 72.08 |
| CML3 | 9.8 | Flongle | 0.09 | 0.03 | 9857 | 19900 | 5860 | 9.45 | 85.54 |
| CML4 | 9.7 | Flongle | 0.93 | 0.28 | 109648 | 15200 | 1800 | 9.31 | 85.97 |
| CML5 | ND | Flongle | 0.04 | 0.03 | 3907 | 22400 | 8090 | 8.95 | 91.06 |
| CML6 | 9.2 | Flowcell | 5.00 | 1.52 | 381552 | 30800 | 7090 | 11.94 | 90.73 |
| AML1 | 9.3 | Flowcell | 5.47 | 1.66 | 639884 | 15100 | 6340 | 9.89 | 86.76 |
| AML2 | 9.2 | Flowcell | 1.59 | 0.48 | 161010 | 19400 | 6540 | 12.00 | 89.95 |
| APL1 | 9.6 | Flowcell | 0.17 | 0.05 | 13486 | 24700 | 7450 | 12.94 | 92.49 |
| APL2 | 7.7 | Flowcell | 1.1 | 0.33 | 40919 | 24200 | 8770 | 12.12 | 92.69 |
| APL3 | 9.7 | Flowcell | 0.31 | 0.09 | 13625 | 22500 | 6910 | 12.42 | 93.51 |
| APL4 | 9.6 | Flowcell | 1.63 | 0.49 | 176665 | 25200 | 3740 | 12.38 | 93.27 |
| APL5 | 9.3 | Flowcell | 0.22 | 0.07 | 31863 | 24200 | 2450 | 11.82 | 91.49 |
| APL6 | 7.5 | Flowcell | 1.06 | 0.32 | 485374 | 4650 | 1230 | 11.70 | 84.27 |

Patient-specific BCR::ABL1 breakpoints were confirmed by performing PCR and Sanger sequencing in 2 cases. Primers were designed using Primer3 v. 0.4.0, described in Krawetz S., Bioinformatics Methods and Protocols: Methods in Molecular Biology, Totowa, NJ Humana Press pp 365-86, which is incorporated herein by reference. 100 ng of DNA were amplified, the PCR product was run on a 2% agarose gel, and Sanger sequenced to confirm the genomic breakpoint. All other patients were confirmed with clinical karyotype and fluorescent in-situ hybridization data. BCR-ABL cell lines breakpoints were confirmed by published data.

The sequence data generated by MinION was processed using a graphical, cloud-based workflow built on Biodepot-workflow-builder (Bwb), in which each computational task is represented by a graphical widget that represents a software container (such as a Docker container) that is executed by a cloud computing system. By executing within containers, each computational task may easily be performed in the cloud, and can readily leverage graphical processing units (GPUs) or other special-purpose processors made available by cloud computing systems. The workflow includes base calling, alignment, fusion gene detection, and visualization. Guppy, version 6.4.6 using the r9.4.1 hac model, was used for base calling. Minimap2 was used for alignment and variant calling. The Integrative Genomics Viewer (IGV) was used for visualization of results, and GRCh37 hg19 was used as the reference genome. QC was performed using PyoQC.

The workflow included detecting fusion genes using the method 900 disclosed above, as well as the previous LongGF technique for the purposes of comparison. LongGF is a software tool for fusion gene detection optimized for the high base calling error rates and alignment errors commonly found in long-read sequencing data. LongGF takes as input a BAM file containing alignments and a GTF file containing the definitions of known fusion genes. The output is a log file with detected gene fusions and their supporting reads.

Empirical experiments were performed to benchmark the sensitivity and runtime required to detect fusion genes. Sequencing metrics, including quality scores and timestamps, were obtained for each sample from the sequencing summary text file, an output of the Guppy base caller. Fusions were detected using both the method 900 described above and LongGF, for comparison. All samples were combined into a dictionary and separated by patient and cell line data. Plots were constructed for each category of data pertaining to the time to detect three fusion genes and the number of reads required to detect three fusion genes. Finally, the total number of fusion gene reads detected for each sample were compared.

Two enrichment metrics were also computed by the workflow and tracked for each sample. First, the fusion-specific enrichment was calculated with the formula [(number of fusion reads)/(mean coverage of the genome)]. Second, the on-target enrichment was calculated with the formula [(number of reads that originate from a guide RNA cut point that includes the region of the breakpoint)/(mean genome coverage)]. Reads originating from a guide RNA cut are distinguished by the guide sequence at the beginning of a read. Initial electrical signal data generated by DNA passing through the nanopores (reads at the beginning of a strand) is error-prone, which affects base calling and, therefore the alignment of the reads. Accordingly, the start sequence is often misaligned. Consequently, the workflow considers base pairs of the sequence near the start (default within 50 bp) of a read sequence that aligns near the coordinates of a guide RNA cut site to have originated from a guide RNA cut. Specific read sequences cut by guides are manually confirmed. The allowed error intervals are customizable. Samtools v1.13 was used to sort and convert BAM files and determine the average coverage. Picard CollectHsMetrics was used to generate the unique base pairs mapped metric for each sample.

Details of the sample sequencing and enrichment metrics are included in Tables 2 and 3. A range of 0.04 Gb-5.47 Gb (gigabases) of sequencing data were generated for each sample for an average mean coverage of the human genome of 0.32-fold (range: 0.01-1.66). Standard quality metrics for nanopore workflows were used and tracked N50, a quality metric where half the reads are above this length (range 4.65 kb-32.2 kb) and median read length for total reads (range 0.87 kb-9.60 kb). The percentage of reads aligned ranged from 72-96%. Fusion-specific enrichment was 135-837-fold for cell lines and 6-509-fold for patient samples. On-target enrichment was 849-5830-fold for cell lines and 535-3007-fold for patient samples.

TABLE 3

| | | Fusion | | Fusion | | |
|---|---|---|---|---|---|---|
| | Fusion | Reads - | | Enrichment | | |
| | Reads - | Method | On-target | Spanning | Fusion Breakpoint | Disease |
| Sample | LongGF | 900 | Enrichment | Breakpoint | Coordinates | Burden |
| K562 | 0 | 29 | 5830.00 | 797.50 | chr9: 133607147; | Cell line |
| | | | | | chr22: 23632742 | |
| KU812 | 0 | 51 | 2108.33 | 467.50 | chr9: 133643198; | Cell line |
| | | | | | chr22: 23632850 | |
| KCL22 | 5 | 171 | 2118.70 | 817.83 | chr9: 133664205; | Cell line |
| | | | | | chr22: 23632116 | |
| NB4 | 3 | 3 | 848.57 | 141.43 | chr15: 74326373; | Cell line |
| | | | | | chr17: 38502175 | |
| MV4; 11 | 57 | 71 | 2698.93 | 836.79 | chr4: 88009085; | Cell line |
| | | | | | chr11: 118353675 | |
| ME1 | 45 | 37 | 1305.49 | 134.18 | chr16: 15815076; | Cell line |
| | | | | | chr16: 67132446 | |
| CML1 | 0 | 142 | 1563.91 | 509.35 | chr9: 133709131; | CML- |
| | | | | | chr22: 23632184 | AP |
| | | | | | | Relapse |
| CML2 | 0 | 4 | 2227.50 | 330.00 | chr9: 133629166; | CML |
| | | | | | chr22: 23632505 | BC |
| CML3 | 0 | 0* | 3006.67 | NA | NA | CML- |
| | | | | | | AP |
| CML4 | 0 | 14 | 557.10 | 49.68 | chr9: 133636186; | CML- |
| | | | | | chr22: 23634109 | CP |
| CML5 | 0 | 8 | 1283.33 | 293.33 | chr9: 133709142; | CML- |
| | | | | | chr22: 23632178 | AP |
| | | | | | | Relapse |
| CML6 | 0 | 106 | 1539.12 | 142.56 | chr9: 133582947; | CML |
| | | | | | chr22: 23633181 | BC |
| AML1 | 28 | 38 | 535.12 | 22.93 | chr9: 20413972; | 90% |
| | | | | | chr11: 118355951 | blasts |
| AML2 | 0 | 0 | 649.62 | NA | NA | 20% |
| | | | | | | blasts |
| APL1 | 0 | 0** | 1242.35 | NA | NA | 0% PB |
| | | | | | | blasts, |
| | | | | | | APL |
| | | | | | | confirmed |
| | | | | | | by |
| | | | | | | FISH on |
| | | | | | | BM |
| | | | | | | sample |
| APL2 | 8 | 10 | 618.00 | 30.00 | chr15: 74316531; | 80% |
| | | | | | chr17: 38489068 | blasts |
| APL3 | 8 | 10 | 872.90 | 106.45 | chr15: 74316604; | 75% |
| | | | | | chr17: 38493994 | blasts |
| APL4 | 0 | 0*** | 1785.64 | NA | NA | 83% |
| | | | | | | blasts |
| APL5 | 0 | 0*** | 2520.00 | NA | NA | 79% |
| | | | | | | blasts |
| APL6 | 0 | 2**** | 650.66 | 6.23 | chr11: 118354923; | 22% |
| | | | | | chr17: 75328696 | blastson |
| | | | | | | flow in |
| | | | | | | a <5% |
| | | | | | | marrow |

Concordance with expected results was 100% for cell lines. The method 900 detected the expected fusion in 6/6 cell lines (3 BCR::ABL1, 1 PML::RARA, 1 CBFB::MYH11, 1 KMT2A::AFF1). Breakpoint sequences detected for BCR::ABL1 cell lines were the same as previously published. The method 900 correctly confirmed the presence or absence of fusion genes in 11/14 (78.5%) primary specimens, including both diagnostic and measurable residual disease (MRD) cases with a minimum of 3 reads. However, one case (APL6) showed only two fusion reads and was not counted as confirmed. The three missed cases (CML3, APL1, and APL6 in Tables 1 and 2) were specimens with low disease burden, ˜0%, 1%, and <5%. The method 900 detected BCR::ABL1 in 5/6 cases, PML::RARA in 2/6 cases, and KMT2A::MLLT3 and CBFB::MYH11 in 1/1 case each. Orthogonal confirmation of the specific breakpoints was conducted for select patient cases via PCR and fusion breakpoints of cell lines were compared to published data. In three specimens from two patients with suspected APL, the method 900 did not detect PML::RARA but observed other findings. Clinical and laboratory details are listed in Table 2. For the first patient, two specimens, one bone marrow and one peripheral blood (APL4 and APL 5), yielded no PML::RARA fusion reads. This patient presented with an AML morphologically suggestive of APL and an isochromosome 17q without t(15;17) detected by karyotype. While fusion detection software did not detect a fusion, manual inspection showed an insertion in an intronic region of RARA with TTMV viral genome. Patient APL6 presented with 22% blasts on flow in a <5% marrow which on unblinding showed a complex karyotype with t (11;17)(q23;q25), including the KMT2A gene. Two reads with KMT2A::SEPTIN9 fusion were detected in a suboptimal but acceptable run (N50<5000 bp; on-target enrichment 650.66 fold), confirming the lack of t(15;17) or PML::RARA fusion, but the threshold was below the requisite three reads to confirm the KMT2A::SEPTIN9 fusion.

In most cell line and primary specimens, LongGF had problems detecting BCR::ABL1 and missed fusion reads that the method 900 identified. Using the method 900, the average total sequencing and data processing time to 3 reads with fusions in the cell line experiments was 42.75 minutes (range: 18.87-77.65 min) and 188 minutes in the primary specimens where three reads were detected (range: 32-654 min) (see Table S1). Cell line experiments took an average of 11,711 reads to identify three fusion reads (range: 1,321-43,326 reads) and 10,273 reads in primary specimens (range: 1,790-24,999 reads) for confirmation of the fusion calls. The method 900 provided precise breakpoints in each read in addition to the number of breakpoints found nearby (see Table 3). In contrast, LongGF only detected and provided breakpoint coordinates when there are enough supporting reads with the same breakpoint.

TABLE S1

| Cell lines | Time to 3 Fusions (min) | Reads to 3 Fusions |
|---|---|---|
| K562 | 51.44 | 2598 |
| KCL22 | 18.87 | 1321 |
| KU812 | 32.16 | 4137 |
| ME1 | 77.65 | 43326 |
| MV411 | 33.65 | 7175 |
| NB4 | 831.26 | 22803 |
| CML1 | 47.33 | 2093 |
| CML2 | 653.56 | 2869 |
| CML3 | NR | NR |
| CML4 | 145.23 | 21201 |
| CML5 | 319.42 | 1790 |
| CML6 | 31.74 | 10303 |
| AML1 | 63.78 | 17414 |
| AML2 | 193.36 | 24999 |
| APL1 | NR | NR |
| APL2 | 98.27 | 9106 |
| APL3 | 136.58 | 2683 |
| APL4 | NR | NR |
| APL5 | NR | NR |
| APL6 | NR | NR |

In five experiments (2 cell lines, three primary specimens), Flongles was used; in the other experiments, MinION flow cells were used. The performance of the affordable Flongles was inferior to the MinION flow cells, with lower expected average data output from the Flongles (based on manufacture expectations of ˜3 GB), the N50 and median read length were smaller in Flongle reads (21,614 vs. 19,166 and 6058 vs. 5250 respectively), and significantly, the median Phred score for Flongle reads was lower than that for the MinION flow cells (9.2 vs. 11.88). Despite the worse performance of the Flongles, fusion genes were detected in 2 of 2 cell lines, and 2 of the three experiments with primary specimens with at least 8 and 14 reads confirming the fusion, and the specimen without fusion confirmation was CML3 with pancytopenia and low disease burden.

It has been determined that there is a need to provide genetic testing that is more portable with a faster turnaround time, testing that can be done closer to the patients, and platforms that can assay several targeted genes simultaneously. To address these issues, embodiments of the present disclosure can be used to detect fusion genes in blood or marrow samples in less than 8 hours. The fastest time to achieve three fusion reads in the example embodiment described above was 5 hours on a portable sequencing device with an accessible cloud-based data analysis workflow. Some features that helped achieve the technical goals were the combining of CRISPR-Cas9 enrichment during library preparation, nanopore long-read sequencing, and a cloud-based data analysis pipeline using the method 900 described above. RNA guides were designed to target genes involved in recurrent fusions in myeloid malignancies and used to enrich an amplification-free library preparation over 1600-fold. The modular and containerized workflow allows users to efficiently process raw FAST5 data on the cloud through an accessible graphical user interface allowing for a very fast analysis step (average ˜4.5 minutes for base calling, alignment, and fusion detection).

To improve fusion calling, the method 900 allows users to identify fusion reads not detected by previous techniques such as LongGF. Published genomic breakpoints were confirmed in the test series of cell lines and archival patient samples, including both diagnostic and follow-up samples, to test feasibility in confirming both common and novel fusions over a range of tumor burdens. The example study included 14 patient specimens and demonstrated the usability of the method 900 in primary specimens with 2 micrograms of DNA.

One advantage of the CRISRP-Cas9 based enrichment protocol is that it allows targeting multiple common leukemia fusion genes by pooling multiple guide RNAs. Fusions are particularly well suited for this technique as they have large gene segments that aid in alignment despite sequencing errors and wide variation where translocation breakpoints may occur. Other labs have employed different methods that detected fusions by targeting one partner gene in the fusion. In contrast, embodiments of the present disclosure target both fusion partners; thus the present disclosure allows for an expanded capability to detect known and novel fusions, such as in the case AML1 with t(9;11), when guides are designed to target t(4;11). While in 2/14 patients the assay did not detect fusion genes, both cases had a very low amount of fusion target. In one case (CML3) because the BCR::ABL1 was <0.01%, the other was in the context of a very hypocellular sample.

The present disclosure differs from standard RNA-based fusion detection assays and instead interrogates single molecules of DNA rapidly and accurately to detect specific translocation breakpoints. Long-read sequencing technologies, like Nanopore (Oxford Nanopore Technologies, Oxford, U.K.), allow the sequencing of unamplified, long unbroken fragments of DNA which are more likely to span a breakpoint. This genomic breakpoint identification has potential clinical utility for personalized disease monitoring when CML patients are on TKI therapy and suppressing RNA transcription, targeting DNA as a monitoring target may be more robust and reproducible since DNA is stable and present in constant numbers. This targeting DNA rather than RNA has higher tolerance for challenged samples, and testing tissues with poor preservation is possible, which would be especially beneficial for LMICs and remote areas that may not have immediate access to a pathology laboratory or even refrigeration. However, genomic breakpoints in BCR::ABL1 are unique to individual patients, which may suggest the use of patient-specific breakpoint characterization as ABL1 breakpoints occur over an expansive region of about 150 Kb, making this an arduous endeavor previously involving multiple primer sets and Sanger sequencing. Embodiments of the present disclosure allow a single approach spanning the BCR and ABL1 breakpoint regions without the use of multiple primers and PCR reactions. Once the breakpoint is known, the sensitivity of DNA-based qPCR can be as low as 10-7. Specimens CML1 and CML5 were a BM and PB (respectively) obtained from the same patient and demonstrate high fidelity in confirming genomic breakpoints and the ability to use patient-specific primers for personalized MRD monitoring.

The advantages of long-read sequencing over current clinical diagnostic assays include speed and the relatively low complexity of the assay when compared to cytogenetics and targeted NGS panels. While long-read sequencing results could potentially have a turnaround time (TAT) of less than 24 hours, full karyotype analysis TAT is generally longer, with the fastest times at days to a week, and most targeted NGS panels require ˜7-10 days from the start of processing to the result report. The nanopore sequencing generates data that can be simultaneously analyzed by the GPU-enabled data analytic pipeline described above, which resides on the cloud to help interpret and reliably identify fusion reads within 5000 seconds (<2 hours) of computational time in most specimens. Three reads were used as a threshold for fusion confirmations (though in other embodiments, other thresholds could be used). With the simultaneous sequencing and data analysis workflow described here, three sequences were detected in an average of 3 hours and 7 minutes (fastest at 30 minutes) in the nine patient specimens where a fusion could be confirmed. Consequently, a diagnostic result with a precise fusion breakpoint with three fusion supporting reads would be possible on the same day.

Cost was also a consideration in developing the workflow described above, as it is desirable for the total cost per assay to be sustainable for patients in LMICs. Five specimens were sequenced on the more affordable Flongle device, which has lower sequencing capabilities with fewer pores and channels but costs around $90 per sequencer. Although the library preparation reagent costs for these early proof of concept experiments were the same (approximately $100), further optimization could lower costs for the Flongle devices. On the Flongles, three fusion reads were reached in 4/5 specimens (2 cell lines, three patient samples). The sequencing quality is significantly lower on the Flongles (median Phred 9.2 in Flongles vs. 11.9 in MinION flow cells). However, fusions were detected in all samples with tumor burden above 5%. It is predicted that additional optimizations of the CRISPR library guides will increase efficiency and on-target fusion reads, which can overcome reduced numbers of pores on the Flongles.

The experiments described above demonstrate the feasibility of using a single molecule long-range sequencing assay to detect fusion genes in patients with heme malignancies (AML, CML and APL), and suggest that detecting fusion genes for other reasons would also be possible. Inherent characteristics of fusion genes make this assay a promising, cost-effective tool for rapid detection of recurrent fusions that 1) require previous knowledge of only one of two target genes as guides can capture an unknown partner gene, 2) has a rapid TAT (8 hours in 80% of samples) when multiplexing different assays and used with the specific data analysis and fusion detection tools, 3) can precisely map translocation genomic breakpoints that allow for development of personalized markers for disease monitoring, and 4) can potentially allow discovery of novel/different fusion partners. The work described above shows that a low-cost, portable fusion cancer diagnostic device with an integrated cloud-based on-demand analysis workflow can be implemented in LMICs. Furthermore, no expensive lab investment or computing infrastructures is needed, thus bridging the gap between diagnosis and tailored treatment administration.

The complete disclosure of all patents, patent applications, and publications, and electronically available material cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern.

The foregoing detailed description and examples have been given for clarity of understanding only. No unnecessary limitations are to be understood therefrom. The disclosure is not limited to the exact details shown and described, for variations obvious to one skilled in the art will be included within the disclosure defined by the claims.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure.

Specific elements of any foregoing embodiments can be combined or substituted for elements in other embodiments. Moreover, the inclusion of specific elements in at least some of these embodiments may be optional, wherein further embodiments may include one or more embodiments that specifically exclude one or more of these specific elements. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, 10 when used in this application, shall refer to this application as a whole and not to any particular portions of the application.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present disclosure. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

All of the references cited herein are incorporated by reference. Aspects of the disclosure can be modified, if necessary, to employ the systems, functions, and concepts of the above references and application to provide yet further embodiments of the disclosure. These and other changes can be made to the disclosure in light of the detailed description.

It will be appreciated that, although specific embodiments of the disclosure have been described herein for purposes of illustration, various modifications may be made without deviating from the spirit and scope of the disclosure. Accordingly, the disclosure is not limited except as by the claims.

As used herein, “phenotype” refers to an appearance of an organism based on a multifactorial combination of genetic traits and environmental factors; a tissue type (e.g., heart tissue vs. adrenal tissue); an organism type (e.g., a strain of bacteria); or an expressed gene.

As used herein, “nanopore” refers to a pore of nanometer size used to generate ionic current changes in response to interactions with molecules present therein.

As used herein, “nucleic acid” refers to a polymer of monomer units or “residues”. The monomer subunits, or residues, of the nucleic acids each contain a nitrogenous base (i.e., nucleobase) a five-carbon sugar, and a phosphate group. The identity of each residue is typically indicated herein with reference to the identity of the nucleobase (or nitrogenous base) structure of each residue. Canonical nucleobases include adenine (A), guanine (G), thymine (T), uracil (U) (in RNA instead of thymine (T) residues) and cytosine (C). However, the nucleic acids of the present disclosure can include any modified nucleobase, nucleobase analogs, and/or non-canonical nucleobase, as are well-known in the art. Modifications to the nucleic acid monomers, or residues, encompass any chemical change in the structure of the nucleic acid monomer, or residue, that results in a noncanonical subunit structure. Such chemical changes can result from, for example, epigenetic modifications (such as to genomic DNA or RNA), or damage resulting from radiation, chemical, or other means. Illustrative and nonlimiting examples of noncanonical subunits, which can result from a modification, include uracil (for DNA), 5-methylcytosine, 5-hydroxymethylcytosine, 5-formethylcytosine, 5-carboxycytosine b-glucosyl-5-hydroxymethylcytosine, 8-oxoguanine, 2-amino-adenosine, 2-amino-deoxyadenosine, 2-thiothymidine, pyrrolo-pyrimidine, 2-thiocytidine, or an abasic lesion. An abasic lesion is a location along the deoxyribose backbone but lacking a base. Known analogs of natural nucleotides hybridize to nucleic acids in a manner similar to naturally occurring nucleotides, such as peptide nucleic acids (PNAs) and phosphorothioate DNA. The five-carbon sugar to which the nucleobases are attached can vary depending on the type of nucleic acid. For example, the sugar is deoxyribose in DNA and is ribose in RNA. In some instances herein, the nucleic acid residues can also be referred with respect to the nucleoside structure, such as adenosine, guanosine, 5-methyluridine, uridine, and cytidine. Moreover, alternative nomenclature for the nucleoside also includes indicating a “ribo” or deoxyrobo” prefix before the nucleobase to infer the type of five-carbon sugar. For example, “ribocytosine” as occasionally used herein is equivalent to a cytidine residue because it indicates the presence of a ribose sugar in the RNA molecule at that residue. A nucleic acid polymer can be or comprise a deoxyribonucleotide (DNA) polymer, a ribonucleotide (RNA) polymer. The nucleic acids can also be or comprise a PNA polymer, or a combination of any of the polymer types described herein (e.g., contain residues with different sugars).

As used herein, “peptide” refers to refers to natural biological or artificially manufactured short chains of amino acid monomers linked by peptide (amide) bonds. As used herein, a peptide has at least 2 amino acid repeating units.

As used herein, “polypeptide” or “protein” refers to a polymer in which the monomers are amino acid residues that are joined together through amide bonds. When the amino acids are alpha-amino acids, either the L-optical isomer or the D-optical isomer can be used, the L-isomers being preferred. The term polypeptide or protein as used herein encompasses any amino acid sequence and includes modified sequences such as glycoproteins. The term polypeptide is specifically intended to cover naturally occurring proteins, as well as those that are recombinantly or synthetically produced. “Protein” can be any of various naturally occurring substances that consist of amino-acid residues joined by peptide bonds, contain the elements carbon, hydrogen, nitrogen, oxygen, usually sulfur, and occasionally other elements (such as phosphorus or iron), and include many essential biological compounds (such as enzymes, hormones, or antibodies).

As used herein, “tissue” refers to an aggregate of similar cells and cell products forming a definite kind of structural material with a specific function, in a multicellular organism.

As used herein, “organ” refers to a group of tissues in a living organism that have been adapted to perform a specific function.

### Examples

The following paragraphs list non-limiting examples of embodiments of the present disclosure.

Example 1. A computer-implemented method of detecting a presence of a predetermined fusion gene in a biological sample, the method comprising: receiving, by a computing system, a read sequence; generating, by the computing system, an alignment of the read sequence to a reference genome, wherein the alignment includes a first alignment result and a second alignment result, wherein the first alignment result indicates an alignment of a first portion of the read sequence to a first location in the reference genome, and wherein the second alignment result indicates an alignment of a second portion of the read sequence to a second location in the reference genome; determining, by the computing system, a breakpoint location indicated by the first alignment result and the second alignment result; determining, by the computing system, distances between coordinates of the breakpoint location and coordinates of one or more expected breakpoint locations associated with the predetermined fusion gene; determining, by the computing system, a gap size value and an overlap size value based on the first portion of the read sequence and the second portion of the read sequence; and in response to determining that the gap size value is less than a gap size value threshold, the overlap size value is less than an overlap size value threshold, and the distances are less than a breakpoint distance threshold, generate an indication of the presence of the predetermined fusion gene.

Example 2. The computer-implemented method of Example 1, wherein the breakpoint location includes a first coordinate associated with the first alignment result and a second coordinate associated with the second alignment result; and wherein determining distances between the coordinates of the breakpoint location and the coordinates of the one or more expected breakpoint locations associated with the predetermined fusion gene includes, for at least one expected breakpoint location: measuring a first distance between the first coordinate associated with the first alignment result and a first coordinate of the at least one expected breakpoint location; and measuring a second distance between the second coordinate associated with the second alignment result and a second coordinate of the at least one expected breakpoint location.

Example 3. The computer-implemented method of any one of Examples 1 or 2, wherein determining the gap size value includes determining a portion of the read sequence that is not included in the first portion of the read sequence or the second portion of the read sequence.

Example 4. The computer-implemented method of any one of Examples 1-3, wherein determining the overlap size value includes determining a portion of the read sequence that is in both the first portion of the read sequence and the second portion of the read sequence.

Example 5. The computer-implemented method of any one of Examples 1-4, wherein receiving the read sequence includes receiving a stream that includes the read sequence from a sequencing device while the read sequence is being generated by the sequencing device.

Example 6. The computer-implemented method of any one of Examples 1-5, wherein the read sequence includes at least 300 bases.

Example 7. The computer-implemented method of Example 6, wherein the read sequence includes at least 1000 bases.

Example 8. The computer-implemented method of any one of Examples 1-7, wherein the computing system includes at least one cloud computing device.

Example 9. The computer-implemented method of any one of Examples 1-8, wherein the computing system includes at least one graphical processing unit (GPU).

Example 10. The computer-implemented method of any one of Examples 1-9, wherein the reference genome excludes portions of a genome of a subject organism that are not associated with the predetermined fusion gene.

Example 11. The computer-implemented method of any one of Examples 1-10, wherein the predetermined fusion gene is PML-RARA, BCR-ABL1 p210, KMT2A-AF4, MYH11-CBFB, or an isoform thereof.

Example 12. The computer-implemented method of any one of Examples 1-11, wherein the reference genome includes at least a portion of a genome of an organism and at least a portion of a genome of a virus.

Example 13. A computer-implemented method of detecting fusion genes in a biological sample, the method comprising: receiving, by a computing system, a plurality of read sequences; for each read sequence of the plurality of read sequences: generating, by the computing system, an alignment of the read sequence to a reference genome, wherein the alignment includes a first alignment result and a second alignment result, wherein the first alignment result indicates an alignment of a first portion of the read sequence to a first location in the reference genome, and wherein the second alignment result indicates an alignment of a second portion of the read sequence to a second location in the reference genome; determining, by the computing system, a breakpoint location indicated by the first alignment result and the second alignment result; determining, by the computing system, a gap size value and an overlap size value based on the first portion of the read sequence and the second portion of the read sequence; and in response to determining that the gap size value is less than a gap size value threshold and the overlap size value is less than an overlap size value threshold, adding, by the computing system, the breakpoint location to a set of candidate breakpoint locations; determining, by the computing system, a similar breakpoint set that includes breakpoint locations of the set of candidate breakpoint locations for which the breakpoint locations are within a threshold distance of each other; and in response to determining that a size of the similar breakpoint set is larger than a threshold, generating an indication of a detected fusion gene associated with the similar breakpoint set.

Example 14. The computer-implemented method of Example 13, wherein each breakpoint location includes a first coordinate associated with the first alignment result and a second coordinate associated with the second alignment result; and wherein distances between the breakpoint locations are determined by, for a first breakpoint location and a second breakpoint location: measuring a first distance between a first coordinate associated with the first breakpoint location and a first coordinate of associated with the second breakpoint location; and measuring a second distance between a second coordinate associated with the first breakpoint location and a second coordinate associated with the second breakpoint location.

Example 15. The computer-implemented method of any one of Example 13 or 14, wherein determining the gap size value includes determining a portion of the read sequence that is not included in the first portion of the read sequence or the second portion of the read sequence.

Example 16. The computer-implemented method of any one of Examples 13-15, wherein determining the overlap size value includes determining a portion of the read sequence that is in both the first portion of the read sequence and the second portion of the read sequence.

Example 17. The computer-implemented method of any one of Examples 13-16, wherein receiving the plurality of read sequences includes receiving a stream that includes read sequences generated by a sequencing device while the read sequences are being generated by the sequencing device.

Example 18. The computer-implemented method of any one of Examples 13-17, wherein the read sequence includes at least 300 bases.

Example 19. The computer-implemented method of Example 18, wherein the read sequence includes at least 1000 bases.

Example 20. The computer-implemented method of any one of Examples 13-19, wherein the computing system includes at least one cloud computing device.

Example 21. The computer-implemented method of any one of Examples 13-20, wherein the computing system includes at least one graphical processing unit (GPU).

Example 22. The computer-implemented method of any one of Examples 13-21, wherein the reference genome includes at least a portion of a genome of an organism and at least a portion of a genome of a virus.

Example 23. A non-transitory computer-readable medium having computer-executable instructions stored thereon that, in response to execution by one or more processors of a computing system, cause the computing system to perform actions of a method as recited in any one of Examples 1-22.

Example 24. A computing system configured to perform actions of a method as recited in any one of Examples 1-22.

1. A computer-implemented method of detecting a presence of a predetermined fusion gene in a biological sample, the method comprising:
receiving, by a computing system, a read sequence;
generating, by the computing system, an alignment of the read sequence to a reference genome, wherein the alignment includes a first alignment result and a second alignment result, wherein the first alignment result indicates an alignment of a first portion of the read sequence to a first location in the reference genome, and wherein the second alignment result indicates an alignment of a second portion of the read sequence to a second location in the reference genome;
determining, by the computing system, a breakpoint location indicated by the first alignment result and the second alignment result;
determining, by the computing system, distances between coordinates of the breakpoint location and coordinates of one or more expected breakpoint locations associated with the predetermined fusion gene;
determining, by the computing system, a gap size value and an overlap size value based on the first portion of the read sequence and the second portion of the read sequence; and
in response to determining that the gap size value is less than a gap size value threshold, the overlap size value is less than an overlap size value threshold, and the distances are less than a breakpoint distance threshold, generate an indication of the presence of the predetermined fusion gene.

2. The computer-implemented method of claim 1, wherein the breakpoint location includes a first coordinate associated with the first alignment result and a second coordinate associated with the second alignment result; and
wherein determining distances between the coordinates of the breakpoint location and the coordinates of the one or more expected breakpoint locations associated with the predetermined fusion gene includes, for at least one expected breakpoint location:
measuring a first distance between the first coordinate associated with the first alignment result and a first coordinate of the at least one expected breakpoint location; and
measuring a second distance between the second coordinate associated with the second alignment result and a second coordinate of the at least one expected breakpoint location.

3. The computer-implemented method of claim 1, wherein determining the gap size value includes determining a portion of the read sequence that is not included in the first portion of the read sequence or the second portion of the read sequence.

4. The computer-implemented method of claim 1, wherein determining the overlap size value includes determining a portion of the read sequence that is in both the first portion of the read sequence and the second portion of the read sequence.

5. The computer-implemented method of claim 1, wherein receiving the read sequence includes receiving a stream that includes the read sequence from a sequencing device while the read sequence is being generated by the sequencing device.

6. (canceled)

7. The computer-implemented method of claim 1, wherein the read sequence includes at least 1000 bases.

8. The computer-implemented method of claim 1, wherein the computing system includes at least one cloud computing device.

9. The computer-implemented method of claim 1, wherein the computing system includes at least one graphical processing unit (GPU).

10. The computer-implemented method of claim 1, wherein the reference genome excludes portions of a genome of a subject organism that are not associated with the predetermined fusion gene.

11. The computer-implemented method of claim 1, wherein the predetermined fusion gene is PML-RARA, BCR-ABL1 p210, KMT2A-AF4, MYH11-CBFB, or an isoform thereof.

12. The computer-implemented method of claim 1, wherein the reference genome includes at least a portion of a genome of an organism and at least a portion of a genome of a virus.

13. A computer-implemented method of detecting fusion genes in a biological sample, the method comprising:
receiving, by a computing system, a plurality of read sequences;
for each read sequence of the plurality of read sequences:
generating, by the computing system, an alignment of the read sequence to a reference genome, wherein the alignment includes a first alignment result and a second alignment result, wherein the first alignment result indicates an alignment of a first portion of the read sequence to a first location in the reference genome, and wherein the second alignment result indicates an alignment of a second portion of the read sequence to a second location in the reference genome;
determining, by the computing system, a breakpoint location indicated by the first alignment result and the second alignment result;
determining, by the computing system, a gap size value and an overlap size value based on the first portion of the read sequence and the second portion of the read sequence; and
in response to determining that the gap size value is less than a gap size value threshold and the overlap size value is less than an overlap size value threshold, adding, by the computing system, the breakpoint location to a set of candidate breakpoint locations;
determining, by the computing system, a similar breakpoint set that includes breakpoint locations of the set of candidate breakpoint locations for which the breakpoint locations are within a threshold distance of each other; and
in response to determining that a size of the similar breakpoint set is larger than a threshold, generating an indication of a detected fusion gene associated with the similar breakpoint set.

14. The computer-implemented method of claim 13, wherein each breakpoint location includes a first coordinate associated with the first alignment result and a second coordinate associated with the second alignment result; and
wherein distances between the breakpoint locations are determined by, for a first breakpoint location and a second breakpoint location:
measuring a first distance between a first coordinate associated with the first breakpoint location and a first coordinate of associated with the second breakpoint location; and
measuring a second distance between a second coordinate associated with the first breakpoint location and a second coordinate associated with the second breakpoint location.

15. The computer-implemented method of claim 13, wherein determining the gap size value includes determining a portion of the read sequence that is not included in the first portion of the read sequence or the second portion of the read sequence.

16. The computer-implemented method of claim 13, wherein determining the overlap size value includes determining a portion of the read sequence that is in both the first portion of the read sequence and the second portion of the read sequence.

17. The computer-implemented method of claim 13, wherein receiving the plurality of read sequences includes receiving a stream that includes read sequences generated by a sequencing device while the read sequences are being generated by the sequencing device.

18. (canceled)

19. The computer-implemented method of claim 13, wherein the read sequence includes at least 1000 bases.

20. The computer-implemented method of claim 13, wherein the computing system includes at least one cloud computing device.

21. The computer-implemented method of claim 13, wherein the computing system includes at least one graphical processing unit (GPU).

22. The computer-implemented method of claim 13, wherein the reference genome includes at least a portion of a genome of an organism and at least a portion of a genome of a virus.

23-24. (canceled)
